(19) United States
(12) Patent Application Publication
(10) Pub. No.: US 2014/0180036 A1
(43) Pub. Date: Jun. 26, 2014

(54) DEVICE AND METHOD FOR PREDICTING AND PREVENTING OBSTRUCTIVE SLEEP APNEA (OSA) EPISODES

(71) Applicant: THE BOARD OF REGENTS FOR OKLAHOMA STATE UNIVERSITY, Stillwater, OK (US)

(72) Inventors: Satish T.S. Bukkapatnam, Stillwater, OK (US); Trung Le, Stillwater, OK (US); Woranat Wongdhamma, Stillwater, OK (US)

(73) Assignee: THE BOARD OF REGENTS FOR OKLAHOMA STATE UNIVERSITY, Stillwater, OK (US)

(21) Appl. No.: 14/137,326

(22) Filed: Dec. 20, 2013

Related U.S. Application Data
(60) Provisional application No. 61/740,970, filed on Dec. 21, 2012.

(51) Int. Cl. A61B 5/00; A61H 23/00; A61M 16/00; A61F 5/56; A61B 5/04; A61B 5/0205; A61G 7/018; A61B 5/0408; A61B 7/04

(52) U.S. Cl. CPC A61B 5/4836; A61B 5/0408; A61B 5/0006; A61B 5/4818; A61H 23/00; A61M 16/0069; A61B 7/04; A61B 5/04012; A61B 5/6805; A61B 5/0205; A61G 7/018; A61F 5/566
USPC 600/301; 600/388; 601/46; 128/204.23; 600/389; 128/845; 128/848

(57) ABSTRACT

A wireless sleep apnea treatment system comprises a garment having at least one ECG monitor, a wireless signal acquisition board in communication with the ECG monitor and the computer and providing the electrical reading from the ECG monitor to the computer, and a patient stimulator controlled by the computer through the wireless signal acquisition board.

### CROSS REFERENCE TO RELATED APPLICATIONS

This application claims the benefit of U.S. Provisional Patent Application Ser. No. 61/740,970, filed on Dec. 21, 2012 and incorporates said provisional patent application by reference into this document as if fully set out at this point.

### STATEMENT REGARDING FEDERAL SPONSORED RESEARCH OR DEVELOPMENT

This invention was made with U.S. Government support under NSF Grant No. IIP-0736485, NSF Grant No. CMMI-0700680, and NSF Grant No. CMMI-1000978 awarded by the National Science Foundation. The Government has certain rights in this invention.

### FIELD OF THE INVENTION

The invention generally relates to systems and methods for predicting and minimizing or averting sleep apnea episodes.

### BACKGROUND

The field of medicine is on the verge of a transformation where healthcare will tend to focus more on the individual in an attempt to prevent illness rather than treat it post-trauma. The systems approach to personalized healthcare is based on integrating concepts of systems biology and medicine known as (P4): personalized, predictive, preventive and participatory medicine [6]. Much of the current P4 emphasis is on collecting physiological data from ECG, CAT scan, genomic data, diet, etc., into large data warehouses and using advanced information infrastructures for predicting and monitoring chronic non-communicable diseases [6, 7]. Of course, early detection of acute disease episodes through noninvasive monitoring has been shown to be effective for patients with chronic disorders if for no other reason than treatment costs tend to escalate exponentially with delay in detection [8].

Among the chronic conditions, of particular concern is obstructive sleep apnea (“OSA”) and sleep-related breathing disorders that affect one fourth of the US population [9]. Several OSA detection and prediction approaches based on correlating the statistical patterns of heart rate, respiration rate, and oxygen saturation (SpO2) signals during OSA episodes have been attempted [10]. For example, spectral energy of intrinsic mode functions have been extracted from empirical mode decomposition of flow rate signals (from a continuous positive airway pressure, or “CPAP”, machine) to estimate likelihood of OSA episodes [11]. Similarly, support vector machines (SVMs) developed using linear, polynomial and radial basis kernel functions, networks, clustering algorithms with wavelet features have been applied to distinguish cases with OSA from those which do not have OSA [12]. Although, considerable attention has been given to OSA detection methods, prediction of (forecast) an impending OSA episode, which is necessary for calibrating CPAP therapy, have not been reported in literature. The few current reported examples (e.g., dynamic belief networks [13, 14]) use limited data from OSA patients to predict OSA episodes, e.g., about 1 sec ahead, or, in some cases, just predict the evolution of the physiological signals (i.e., heart rate, chest volume, blood oxygen saturation). These methods do not capture variations in nonlinear and nonstationary dynamics of the cardiorespiratory system responsible for the onset of OSA or sleep-related breathing disorder events.

Of course, in practice predictive measurements rely on real-time or near real-time biometric data which must be gathered from the subject. However, the sensors that are required to collect such information often interfere with the patient's ability to sleep, thereby compounding the problem that predictive approaches seek to remedy. More particularly, the development of a wearable multisensory unit that would facilitate gathering of signals necessary of prediction without causing palpable discomfiture remains elusive.

Heretofore, as is well known in the sleep apnea field there has been a need for an invention to address and solve the disadvantages of prior art methods. Accordingly, it should now be recognized, as was recognized by the present inventors, that there exists, and has existed for some time, a very real need for a system and method that would address and solve the above-described problems.

Before proceeding to a description of the present invention, however, it should be noted and remembered that the description of the invention which follows, together with the accompanying drawings, should not be construed as limiting the invention to the examples (or preferred embodiments) shown and described. This is so because those skilled in the art to which the invention pertains will be able to devise other forms of the invention within the ambit of the appended claims.

### SUMMARY OF THE INVENTION

The invention of the present disclosure, in one aspect thereof, comprises a wireless sleep apnea treatment system comprising a computer, a garment having at least one ECG monitor embedded therein in a position to take an electrical reading from a patient's heart when the garment is worn, a wireless signal acquisition board in communication with the ECG monitor and the computer and providing the electrical reading from the ECG monitor to the computer, and a patient stimulator controlled by the wireless signal acquisition board that stimulate the patient in response to a command from the computer upon a predetermined condition being observed by the computer in the electrical reading.

In some embodiments the patient stimulator is a vibrator affixed to the garment and the garment may be a jacket. The patient stimulator may also comprise a CPAP machine, an oral appliance, or an adjustable bed.

In some embodiments the system further comprises a heart sound sensor embedded in the garment and coupled to the wireless signal acquisition board for providing heart sound data to the computer. The computer may utilize a single ECG lead to determine the predetermined condition. The predetermined condition may be a forthcoming sleep apnea event.

The invention of the present disclosure, in another aspect thereof, comprises a wearable sensor vest for use in treatment of sleep apnea. The vest comprises a vest body configured to rest in a predetermined position on a patient's torso, at least one ECG sensor affixed to the vest body in a predetermined position suitable for taking at least one ECG lead reading, a wireless signal acquisition board communicatively coupled to the ECG sensor, and a patient stimulator communicatively coupled to the wireless signal acquisition board. The wireless signal acquisition board provides wireless communication to a control computer and reports information from the ECG sensor thereto, and the wireless signal acquisition board activates the patient stimulator in response to a command from the computer.

In some embodiments the vest further comprises a heart sound sensor affixed to the vest body in a predetermined position suitable for detecting the patient's heartbeat, the heart sound sensor providing heart sound data to the wireless signal acquisition board for relay to the computer. The patient stimulator may be a vibrator affixed to the vest body, and may affix to the vest body so as to be in proximity to the patient's neck when the vest is worn. In some embodiments, the wireless signal acquisition board reports only a single ECG lead data to the computer.

The invention of the present disclosure, in another aspect thereof, comprises a method of modeling sleep apnea events including using a sensor vest affixed to a patient to determine a heart rate of a patient, determining a respiration rate of the patient based on the heart rate, and determining an impeding obstructive sleep apnea episode based on the heart rate and respiration using a Dirichlet process-Gaussian Mixture model.

In some embodiments, for a dataset (x, y), where the x is the historic realizations of a signal, and y is the signal for a future time, mixture of expert model can be expressed as p(y|x,θ)=Σ_{c}p(y|x, c, θ)p(c|x, Φ) where c=(c₁, c₂, . . . ,cₙ) is a discrete indicator that assigns data points to experts whose number is defined using a Dirichlet process, and θ=(θ₁, θ₂, . . . , θₙ) represents the hyperparameters for each Gaussian expert. In further embodiments, G₀ is defined to be a distribution over θ, and α₀ is a positive real scalar and distribution G₀ is Dirichlet process distributed as

G˜DP(α₀, G₀), if for any k partitions {A₁, A₂, . . . , Aₖ} of θ,

(G(A₁), . . . ,G(Aₖ))˜Dir(α₀G₀(A₁), . . . , α₀G₀(Aₖ)).

### BRIEF DESCRIPTION OF THE DRAWINGS

Other objects and advantages of the invention will become apparent upon reading the following detailed description and upon reference to the drawings in which:

FIG. 1 illustrates an embodiment of the invention.

FIG. 2 illustrates a high level system diagram of an embodiment suitable for use with the instant OSA episode prediction invention.

FIG. 3 contains screenshots of 3-channel streaming VCG, 3-D color coded dynamic VCG, and 12-lead transformed ECG signals collected according to the instant invention.

FIG. 4 contains KS statistic variations of extracted features. KS statistic indicates the maximal feature distribution differences between sleep apnea and non-apnea groups.

FIG. 5 contains observation from 300^{th} to 380^{th} min and multiple step-ahead predictions from 341^{th} to 380^{th} min of sleep apnea status, LVM, and NPSD features from patient a05.

### DETAILED DESCRIPTION

According to one aspect of the invention, there is provided a system and method for using data gathered from a user to predict the occurrence of OSA events, which events can then be averted by automatic adjustment of a sleep position to prevent or minimize collapse of the airways.

In some embodiments, a wireless wearable multisensory suite is used to synchronously gather multiple heterogeneous signals, including VCG, ECG, sound, cardiac and respiration in real-time during sleep. Quantifiers of the coupled nonlinear and nonstationary cardiorespiratory dynamics underlying the measured signals are used as inputs to predict the onset of sleep apnea events. In other embodiments, a wearable multisensory wireless unit will be used that is customizable to the specific conditions of the patient such as age, gender, BMI, and diseases.

According to one aspect of the invention, there is provided a method based on using data gathered from a wireless wearable multisensory suite to predict the occurrence of sleep apnea events which are used for automatic adjustment of the sleep position and that avert the collapse of the airways. A unique wireless wearable multisensory suite to synchronously gather multiple heterogeneous signals, including VCG, ECG, sound and respiration has been developed to continuously collect the cardiac and respiratory signals in real-time during sleep. Quantifiers of the coupled nonlinear and nonstationary cardiorespiratory dynamics underlying the measured signals are used as the inputs to predict the onset of sleep apnea events.

Among the main aspects of the present disclosure are (a) a method to provide accurate prediction of an impending OSA episode by considering the nonlinear and nonstationary cardiorespiratory dynamics underlying the measured signals, and (b) the development of an economical wearable wireless multisensor and actuation unit capable of measuring signals useful for sleep monitoring, including ECG, heart sound, respiration without causing significant discomfort or constraints on motion, and (c) an OSA prediction and prevention system that modifies the upper airway by gradually changing the posture of the mandible, tongue or the body.

The foregoing has outlined in broad terms the more important features of the invention disclosed herein so that the detailed description that follows may be more clearly understood, and so that the contribution of the instant inventors to the art may be better appreciated. The instant invention is not limited in its application to the details of the construction and to the arrangements of the components set forth in the following description or illustrated in the drawings. Rather, the invention is capable of other embodiments and of being practiced and carried out in various other ways not specifically enumerated herein. Additionally, the disclosure that follows is intended to apply to all alternatives, modifications and equivalents as may be included within the spirit and the scope of the invention as defined by the appended claims. Further, it should be understood that the phraseology and terminology employed herein are for the purpose of description and should not be regarded as limiting, unless the specification specifically so limits the invention.

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

According to a first aspect of the instant invention there is provided an OSA prediction system. According to another embodiment, there is a prevention system that utilizes the prediction system to prevent OSA events from occurring.

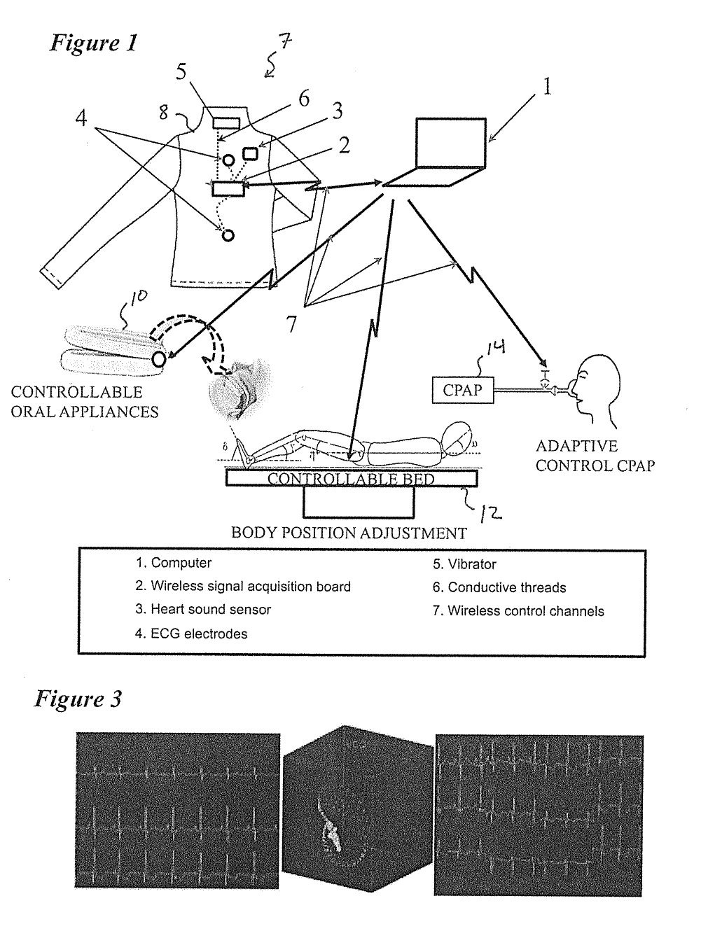

FIG. 1 contains a schematic illustration of a hardware configuration of the instant invention. A computer 1 may be in communication with one or more other devices such as a wearable vest 7 with a body 8 containing, or otherwise having affixed thereto, a wireless signal acquisition board 2, a heart sound sensor 3, a set of ECG electrodes 4, a vibrator 5, all of which may be interconnected with a series of conductive or communicative threads or wires 6. In some embodiments, the computer 1 may be in communication with a controllable oral appliance 10, a controllable bed 12, and/or an adaptive control CPAP device 14.

The computer 1 may contain the requisite programming, software, and routines to implement the control and prediction methods described below. The computer 1 may comprise a personal computer, a laptop, a tablet, a smartphone, or other device capable of being programmed to perform general computing and control of other devices. In some embodiments, the computer 1 communicates with external devices wirelessly.

The vest 7 may be a form fitting, but comfortable garment that may be placed on a patient or user such that the head sound sensor 3 and ECG sensors 4 are properly located on the patient torso to accurately record and report measurements to the computer 1 via the wireless signal acquisition board 2. As described below, the computer 1 will use the gathered data to make predictions concerning impending sleep apnea events.

In order to correct or prevent a detected or predicted sleep apnea event, the computer 1 may electronically control a number of devices to provide a stimulus to the patient. In the case of a patient wearing the vest 7, the computer may wirelessly signal the vest 7 to activate the vibrator 5 for a predetermined period of time. If the oral appliance 10 is used, it may be activated or controlled by the computer 1. Similarly, if the patient is on the adjustable bed 12, the position of the patient may be altered. The computer 1 may also be configured to interface with and/or control a CPAP machine 14.

A first embodiment of the instant invention will utilize a multisensory platform to synchronously gather multiple heterogeneous signals, including ECG, heart sound, etc., and to wirelessly transmit the collected data to a host computer for on-line OSA prediction and subsequent therapeutic decision support. Such multi-channel data would be useful to track dynamic decouplings known to precede transitions that lead to emergence of OSA episodes.

Aspects of an embodiment of a multi-sensor unit will include:
(1) The sensors will be judiciously chosen to capture the complementary aspects of the heart operation, viz. electrical (ECG), acoustic (sound) and mechanical (respiration).
(2) Due to the use of MEMS (Microelectromechanical systems) technology, the total footprint of the wireless unit will be highly adjustable and remains lightweight, and hence highly wearable.
(3) The hardware platform in this context will contribute towards affordable, yet powerful, early warning (prognostic) systems for sleep apnea treatment.
(4) The wireless (Bluetooth) platform along with the sensors and microprocessor components will be integrated into a customized garment to continuously monitor and predict sleep apnea episodes [15].
(5) Multiple step (e.g., minutes) ahead prediction will be used to control OSA devices that can be used to prevent the occurrence of an OSA episode.
(6) The prediction results can be used to adjust the flow rate and other parameters of CPAP devices, or body or positions in an adjustable bed. Alternatively, the prediction can be used to provide a gentle stimulation to the throat (tracheal and laryngeal) muscles to prevent the constriction of the respiratory tract.

In one embodiment, the wireless design will utilize a class I Bluetooth device with response frequency range of 0.176-90 Hz, sampling rate up to 2 Khz, and resolution of 16 bit. Embedded of multiple sensors as part of the garment and the fusion of information from VCG, heart sound and respiration will provide adequate information to track variations and detect transitions in cardiorespiratory dynamics during sleep.

In one embodiment, an ECG lead (e.g., Lead II) is the sole sensor. Heart rate signals are derived from ECG using R-peak identification methods. Respiration rate signal is derived using empirical mode decomposition, wavelet and Fourier filtering methods. Features extracted from heart rate, and respiration are used to predict OSA.

Different OSA treatment devices utilizing the prediction results to timely prevent occurrences of OSA episodes include, without limitation: CPAP airflow adjustment, oral appliances, body position adjustment bed, and noninvasive wearable vibrator.

According to one embodiment of the invention, a Dirichlet process-Gaussian Mixture (DPGM) is used as a model to predict the complex evolution of the OSA signatures. For a dataset (x, y), where the x is the historic realizations of a signal, and y is the signal for a future time, mixture of expert model can be expressed as

$$p ( y \vert x , θ ) = {\sum }_{c}^{ } p ( y \vert x , c , θ ) p ( c \vert x , φ )$$

where c=(c₁, c₂, cₙ) is the discrete indicator that assigns data points to experts whose number is defined using a Dirichlet process, and θ=(θ₁, θ₂, . . . , θₙ) represents the hyperparameters for each Gaussian expert. Here one may define G₀ to be a distribution over θ, and α₀ is a positive real scalar. Distribution G₀ is Dirichlet process distributed as

G˜DP(α₀, G₀), if for any k partitions {A₁, A₂, . . . , Aₖ} of θ,

(G(A₁), . . . , G(Aₖ))˜Dir(α₀G₀(A₁), . . . , α₀G₀(Aₖ)).

The Dirichlet process can be used to extend a mixture model with a countably infinite number of components. In one embodiment, the conditional probability of a single indicator when integrating over the πⱼ variables and letting k tend to infinity is given as:

$${w}_{j} = p ( {c}_{i} = j \vert {c}_{- i} , α ) = \frac{{n}_{- i , j}}{n - 1 + α}$$

$${w}_{n} = p ( {c}_{i} \neq {c}_{j} ∀ j \neq i \vert {c}_{- i} ) = \frac{α}{N - 1 + α}$$

for an existing cluster (expert) and a new expert respectively. Here, n_{—i,j} is the occupation number of expert j excluding observation i, and n is the total number of data points. The parameter can be found by adaptive rejection sampling algorithm [16]. The assigning probability plays the role of weight for each expert. Given a new input x., it is possible to obtain local predictions y̅ₖ (k=1,2, m) from each segment using GP (Gaussian process) formula, then the output for input x, can be expressed as a weighted average

$${y}_{*} = \frac{{\sum }_{k = 1}^{m} {w}_{k} {\overline{y}}_{k}}{{\sum }_{k = 1}^{m} {w}_{k}}$$

This is the least squares fit to the weighted m local predictions.

For multi-step prediction, after the first step, the input to the LGP model is random, which follows a Gaussian distribution, as obtained from previous-step prediction. It will be assumed, in this embodiment, that the input x.˜N (μ_{x.}, Σ_{x.}), where μ_{x.} and Σ_{x.} can be obtained from the equation for p(y|x, θ), supra. The output distribution is given by

P(ƒ(x.)|μ_{x.}, Σ_{x.}, X,Y)=ƒP(ƒ(x.)|x., X, Y)P(x.)dx.

Since it may be difficult to obtain the analytic solution for y., a Monte Carlo approach can be used in this embodiment to approximate the integration, as shown below:

$$P ( f ( {x}_{*} ) \vert {μ}_{{x}_{*}} , {{\sum }_{{x}_{*}}}^{ } , X , Y ) = \frac{1}{N} {\sum }_{t = 1}^{N} P ( f ( {x}_{*}^{t} ) \vert {x}_{*}^{t} , X , Y ) ,$$

where N is the total number of samples.

In a preferred arrangement, the predictive equations used herein were developed from two sources of data: data collected from the Apnea-ECG Database—Physionet.org and from a wireless multisensory platform develop by COMMSENS (OkState) lab. The first source of data was 35 recordings including a continuous digitalized ECG signal sampled at 100 Hz, 16 bits resolution, and a set of apnea annotations in minute wide. The annotations of sleep apnea are made by human expert based on supplementary signals including chest and abdominal respiratory effort signals, oronasal airflow, oxygen saturation. The second source of data was 20 recordings from healthy male subjects (25-35 age ranges), each record containing 3 channels of VCG signal, heart sound, and respiratory signal. The data were sampled at 250 Hz, 16 bit resolutions. These signals were collected from the subjects undergoing different conditions including: rest sitting, upright standing, under a problem solving test, and after exercise. These two sources of data are used for the validation of the research as described in the example that follows.

### General Discussion:

Some aspects of the instant invention include (a) the development of an economical wearable wireless multisensory unit capable of measuring signals essential for sleep monitoring, including ECG, heart sound, respiration, and SPO2 synchronously without causing posing significant discomfort or constraints on motion, and (b) a method to provide accurate prediction of an impending OSA episode by considering the nonlinear and nonstationary cardiorespiratory dynamics underlying the measured signals and the features extracted therefrom.

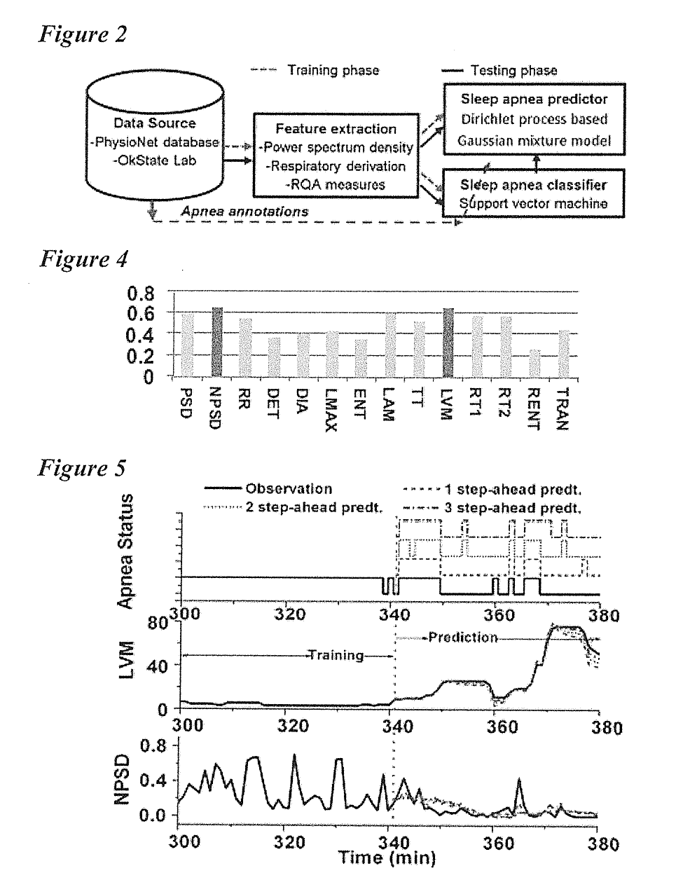

As summarized in FIG. 2, the data from sleep apnea-ECG database is used as well as signals gathered from a wearable multisensory unit for training and testing of the predictor and classifier. While the PhysioNet database consists of signals gathered from chronic OSA patients, in developing the instant methodology the signals from the wearable multisensory unit were gathered from healthy subjects (to assess false positive rates). Various quantifiers of topology of the nonlinear attractor of cardiorespiratory dynamics reconstructed from the measured signals, including laminarity, determinism, entropy, recurrence rate were extracted as features θ to identify an OSA event using a support vector machine (SVM) classifier. The evolution of θ(t) was tracked using a nonparametric Dirichlet process based Gaussian mixture (DPMG) prediction method that effectively captures the nonlinear nonstationary evolution of cardiorespiratory dynamics, which in turn is responsible for the onset of OSA events and other sleep-related breathing disorder episodes.

The k-step (minutes) look-ahead predictions \hat{θ} (t+k) of feature values were used to detect an impending OSA episode 1-3 minutes earlier with an accuracy of 70-90%. Such predictions can be vital to initiate adjustments or therapeutic interventions to avert an impending OSA episode [15].

### Wireless Wearable Multisensory Platform Embodiment:

In one embodiment, a multisensory platform was developed for use in synchronously gathering multiple heterogeneous signals, including VCG, ECG, sound and respiration, (for example, see FIG. 3 for screenshot of real-time streaming VCG, 3-D color coded VCG, and a standard display of 12-lead derived ECG), and wirelessly transmit the data to a host computer for on-line OSA prediction and subsequent therapeutic decision support. Such multi-channel data would be necessary to track dynamic decouplings known to precede transitions that lead to emergence of OSA episodes. Exemplary novel aspects of the multi-sensor unit are as follows: (1) the sensors are judiciously chosen to capture the complementary aspects of the heart operation, viz. electrical (ECG), acoustic (sound) and mechanical (respiration). (2) Due to the use of MEMS technology, total footprint of the wireless unit is highly adjustable and remains lightweight, and hence highly wearable. (3) The hardware platform in this context contributes towards affordable, yet powerful, early warning (prognostic) systems for sleep apnea treatment. (4) The wireless (Bluetooth) platform along with the sensors and microprocessor components are integrated into a customized garment to continuously monitor and predict sleep apnea episodes [16]. In this embodiment, the wireless design utilizes a class I Bluetooth device with response frequency range of 0.176-90 Hz, sampling rate up to 2 Khz, and resolution of 16 bit. Embedded of multiple sensors as part of the garment and the fusion of information from VCG, heart sound and respiration provide adequate information to track variations and detect transitions in cardiorespiratory dynamics during sleep. (5) Single lead (lead II) of ECG can be used to generate the necessary signals, namely the heart rate and respiration rate signals for feature extraction. In an embodiment, an R-peak identification method is used to generate the heart rate and empirical mode decomposition method is used to derive the respiration.

### Feature Extraction:

In one embodiment, feature extraction is performed as is discussed below. Note that the discussion that follows is intended to provide a specific example of an embodiment of the invention and should not be used to limit its practice or the scope of the claims that follow.

In this example, first a band-pass filter with a pass band in the range of 0.06-40 Hz was employed to remove the noise, artifacts, base-line wandering and retain the critical features for the R peak extraction from VCG signals. After de-noising, R peaks of the ECG signal were detected by using the wavelet transformation. The heart rate time series known as RR intervals was then calculated as the time difference between the consecutive R peaks. Abnormal heart rates defined as its amplitude is 80% higher than the previous heart rate will be eliminated from RR intervals. Power spectral density (PSD) analysis of the RR intervals in low frequency band (0.04 to 0.12 Hz) used to capture the heart rate variability in OSA patients. The PSD time series is formulated such that each point is the average power spectral density of one minute of RR interval time series. The normalized PSD (NPSD) feature is considered to account for the inter-subject variability.

Recurrence quantification analysis (RQA) was employed in this example to capture the nonlinear and nonstationary characteristics of the RR interval signals. Time delay ζ=5 was determined based on a mutual information test [18] and dimension d=7 was based on the false nearest neighbors test [19] were used to reconstruct the phase space. The threshold of the recurrent plot is identified as 10% of the maximum phase space diameters. The RQA features are extracted based on sliding window concept with the window's size of 600 data points and the sliding step of 60 data points corresponding to 10 min length and 1 min step of the RR interval time series, respectively. The 10 min size of each sliding window is selected to accommodate the whole longest sleep apnea episode that the patient might experience. The sliding step of 60 sec is sufficient to characterize the cyclic variance of the heart rate which ranges from 20 to 60 sec. Recurrence features extracted from the each window quantify for the complex structures of the recurrence plot of 10 min RR interval. The extracted features from the recurrence plot are recurrence rate (RR), determinism (DET), length of the longest diagonal line (LMAX), entropy (ENT), laminarity (LAM), trapping time (TT), length of longest vertical line (LVM), recurrence time of 1st type (RT1), recurrence time of 2nd type (RT2), recurrence period entropy density (RENT), and transitivity (TRAN). Further details of these quantifiers may be gathered from Marwan's paper [7].

### Classification Model:

A nonlinear support vector machine classification model was employed to determine the sleep apnea events based on the PSD features and RQA extracted features. To reduce the high dimensionality of input space (14 features), KS tests were performed to select the most significant features that effectively classify the input space into sleep apnea and non-apnea groups. FIG. 4 shows the KS statistic value of 14 features. Two significant features with the highest KS statistic—normalized PSD and LVM were selected as the inputs of the classifier. Using a K-fold training and cross validation process the accuracy of the offline OSA classification was determined to be 88%.

### Prediction Results:

Based on the example procedures set out above, a determination was made as to the prediction assurance of the instant method using different models. A summary of those results may be found in Table 1 below.

TABLE 1
COMPARISON OF THE PREDICTION ACCURACIES OF DIFFERENT
MODELS FOR UP TO 3 MIN LOOK-AHEAD PREDICTIONS

| | R² | Classification |
|---|---|---|
| | (first/last | accuracy |
| Method | Step) | (first/last step) |
| ARMA | 0.37/0.1 | 0.4/0.03 |
| EMD | 0.45/0 | 0.67/0.53 |
| DPMG | 0.92/0.51 | 0.83/0.77 |

Among the prediction methods tested in this embodiment, DPMG yields the highest R² and classification accuracy for different prediction horizon as summarized in Table 1 supra. Here the performance of the feature predictions was investigated using the R² the performance of overall forecasting by the classification accuracy using the predicted values. It is noted that the performances of DPMG is better than the classical prediction ARMA model both in prediction and classification performance. Furthermore, with the increasing of the prediction horizon the accuracy of DPMG model does not drop significantly. FIG. 5 shows the training and prediction data of the LVM, NPSD features and the sleep apnea status with the prediction point started at the 341^{th} min. It is observed that DPMG model with different prediction horizons can capture the trend and the amplitude of the observation features thus yields reasonable high prediction accuracies of apnea conditions (i.e., 83% for 1 step-ahead prediction and 77% for 3 step-ahead prediction).

### Conclusions

An approach has been developed that consists of a wearable wireless multisensory platform and uses a novel prediction method to provide 1-3 minutes ahead early warning of an impending sleep apnea episode. This wearable wireless multisensory system can serve as a viable effective platform to continuously and noninvasively acquire physiological signals to track cardiorespiratory dynamics, and quantitatively assess apneic conditions for prediction of OSA episodes. Extensive testing with multiple recordings from Physionet database and the wearable multisensory unit suggests that the classification and prediction accuracies (R²) of 70-90% are possible from the present approach. It was also evident during testing of one variation of the instant invention that the longest vertical length of the recurrence plot and normalized power spectral density are the most sensitive features for OSA episode prediction with offline OSA classification accuracy of 88%. Pertinently, DPMG was shown to provide OSA prediction accuracy of 83% (20-40% more than other methods tested) 1 step-ahead and 77% for 3 step-ahead. Such early prediction is necessary to spur the development of adaptive flow control systems for CPAP devices and induce minor adjustments to body positions to mitigate OSA.

Insofar as the description herein and the accompanying drawings disclose any additional subject matter that is not within the scope of the claim(s) below, the subject matter related to such inventions are not dedicated to the public and the right to file one or more applications to claim such additional inventions is hereby reserved.

Additionally aspects of the instant invention may be disclosed in one or more appendices hereto. Applicants hereby incorporate by reference into this disclosure the contents of any and all of such appendices, as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those skilled in the art, without departing from the spirit of the inventive concept the scope of which is to be determined by the following claims.

### References

[1] T. Young et al., “The Occurrence of Sleep-Disordered Breathing among Middle-Aged Adults,” New England Journal of Medicine, vol. 328, 1230-1235, 1993.
[2] P. Lavie, et al., “All-Cause Mortality in Males with Sleep Apnoea Syndrome: Declining Mortality Rates with Age,” European Respiratory Journal, vol. 25, 514-520, 2005.
[3] L. S. Doherty, et al., “Long-Term Effects of Nasal Continuous Positive Airway Pressure Therapy on Cardiovascular Outcomes in Sleep Apnea Syndrome,” CHEST Journal, vol. 127, 2076-2084, 2005.
[4] J. M. Marin, et al., “Long-Term Cardiovascular Outcomes in Men with Obstructive Sleep Apnoea-Hypopnoea with or without Treatment with Continuous Positive Airway Pressure: An Observational Study,” The Lancet, vol. 365, 1046-1053, 2005.
[5] T. E. Weaver and R. R. Grunstein, “Adherence to Continuous Positive Airway Pressure Therapy,” Proceedings of the American Thoracic Society, vol. 5, 173-178, 2008.
[6] L. Hood, et al., “Systems Biology and New Technologies Enable Predictive and Preventative Medicine.,” Science, vol. 306, 640-643, Oct 22 2004.
[7] Q. Tian, et al., “Systems Cancer Medicine: Towards Realization of Predictive, Preventive, Personalized, and Participatory (P4) Medicine,” Journal of Internal Medicine, vol. 271, 111-121, 2012.
[8] R. Snyderman and Z. Yoediono, “Prospective Care: A Personalized, Preventative Approach to Medicine.,” Pharmacogenomics, vol. 7, 5-9, Feb 2006.
[9] J. E. Stahmann, et al., “Prediction of Disordered Breathing,” United States Patent U.S. Pat. No. 7,938,782 B2, 2011.
[10] T. Penzel, et al., “Systematic Comparison of Different Algorithms for Apnoea Detection Based on Electrocardiogram Recordings,” Medical and Biological Engineering and Computing, vol. 40, 402-407, 2002.
[11] M. O. Mendez, et al., “Automatic Screening of Obstructive Sleep Apnea from the Ecg Based on Empirical Mode Decomposition and Wavelet Analysis,” Physiol. Meas., vol. 31, 273-289, 2010.
[12] O. Fontenla-Romero, et al., “A New Method for Sleep Apnea Classification Using Wavelets and Feedforward Neural Networks,” Artif Intell. Med., vol. 34, 65-76, May 2005.
[13] P. Dagum and A. Galper, “Time Series Prediction Using Belief Network Models,” International Journal of Human-Computer Studies, vol. 42, 617-632, 1995.
[14] J. Bock and D. A. Gough, “Toward Prediction of Physiological State Signals in Sleep Apnea,” IEEE Transactions on Biomedical Engineering, vol. 45, 1332-1341, 1998.
[15] E. D. Weitzman, et al., “Quantitative Analysis of Sleep and Sleep Apnea before and after Tracheostomy in Patients with the Hypersomnia-Sleep Apnea Syndrome,” Sleep, vol. 3, 407-423, 1980.
[16] S. Bukkatpatnam, et al., “Healthsmart Garment Design: A Method for Integrating Multiple Wireless Mems Sensors into a Smart Garment,” 2011.
[17] W. R. Gilks and P. Wild, “Adaptive Rejection Sampling for Gibbs Sampling,” Journal of the Royal Statistical Society. Series C (Applied Statistics), vol. 41, 337-348, 1992.
[18] A. M. Fraser and H. L. Swinney, “Independent Coordinates for Strange Attractors from Mutual Information,” Physical Review A, vol. 33, 1134-1140, 1986.
[19] M. B. Kennel, et al., “Determining Embedding Dimension for Phase-Space Reconstruction Using a Geometrical Construction,” Physical Review A, vol. 45, 3403-3411, 1992.

What is claimed is:

1. A wireless sleep apnea treatment system comprising:
a computer;
a garment having at least one ECG monitor embedded therein in a position to take an electrical reading from a patient's heart when the garment is worn;
a wireless signal acquisition board in communication with the ECG monitor and the computer and providing the electrical reading from the ECG monitor to the computer; and
a patient stimulator controlled by the wireless signal acquisition board that stimulate the patient in response to a command from the computer upon a predetermined condition being observed by the computer in the electrical reading.

2. The system of claim 1, wherein the patient stimulator is a vibrator affixed to the garment.

3. The system of claim 2, wherein the garment is a jacket.

4. The system of claim 1, wherein the patient stimulator comprises a CPAP machine.

5. The system of claim 1, wherein the patient stimulator comprises an oral appliance.

6. The system of system of claim 1, wherein the patient stimulator is an adjustable bed.

7. The system of claim 1, further comprising a heart sound sensor embedded in the garment and coupled to the wireless signal acquisition board for providing heart sound data to the computer.

8. The system of claim 1, wherein the computer utilizes a single ECG lead to determine the predetermined condition.

9. The system of claim 1, wherein the predetermined condition is a forthcoming sleep apnea event.

10. A wearable sensor vest for use in treatment of sleep apnea comprising;
a vest body configured to rest in a predetermined position on a patient's torso;
at least one ECG sensor affixed to the vest body in a predetermined position suitable for taking at least one ECG lead reading;
a wireless signal acquisition board communicatively coupled to the ECG sensor; and
a patient stimulator communicatively coupled to the wireless signal acquisition board;
wherein the wireless signal acquisition board provides wireless communication to a control computer and reports information from the ECG sensor thereto; and
wherein the wireless signal acquisition board activates the patient stimulator in response to a command from the computer.

11. The vest of claim 10, further comprising a heart sound sensor affixed to the vest body in a predetermined position suitable for detecting the patient's heart beat, the heart sound sensor providing heart sound data to the wireless signal acquisition board for relay to the computer.

12. The vest of claim 10, wherein the patient stimulator comprises a vibrator affixed to the vest body.

13. The vest of claim 12, wherein the vibrator affixes to the vest body so as to be in proximity to the patient's neck when the vest is worn.

14. The vest of claim 10, wherein the wireless signal acquisition board reports only a single ECG lead data to the computer.

15. A method of modeling sleep apnea events comprising:
using a sensor vest affixed to a patient to determine a heart rate of a patient;
determining a respiration rate of the patient based on the heart rate; and
determining an impeding obstructive sleep apnea episode based on the heart rate and respiration using a Dirichlet process-Gaussian Mixture model.

16. The method of claim 15, wherein for a dataset (x, y), where the x is the historic realizations of a signal, and y is the signal for a future time, mixture of expert model can be expressed as
$$p ( y \vert x , θ ) = {\sum }_{c} p ( y \vert x , c , θ ) p ( c \vert x , φ )$$
where c=(c₁, c₂, cₙ) is a discrete indicator that assigns data points to experts whose number is defined using a Dirichlet process, and θ=(θ₁, θ₂, . . . , θₙ) represents the hyperparameters for each Gaussian expert.

17. The method of claim 15, wherein G₀ is defined to be a distribution over θ, and α₀ is a positive real scalar and distribution G₀ is Dirichlet process distributed as
G˜DP(α₀, G₀), if for any k partitions {A₁, A₂, . . . , Aₖ} of θ,
(G(A₁), . . . , G(Aₖ))˜Dir(α₀G₀(A₁), . . . , α₀G₀(Aₖ)).
